(19) United States
(12) Patent Application Publication
(10) Pub. No.: US 2015/0359469 A1
(43) Pub. Date: Dec. 17, 2015

(54) Device for Detecting Blood Analytes Transdermally

(71) Applicants: Mark Jacobs, Miami Beach, FL (US); Menachem Mendel Fellig, Miami Beach, FL (US)

(72) Inventors: Mark Jacobs, Miami Beach, FL (US); Menachem Mendel Fellig, Miami Beach, FL (US)

(21) Appl. No.: 14/742,440

(22) Filed: Jun. 17, 2015

Related U.S. Application Data
(60) Provisional application No. 62/013,399, filed on Jun. 17, 2014.

(51) Int. Cl. A61B 5/1477; A61B 5/00; A61B 5/145

(52) U.S. Cl. CPC A61B 5/1477; A61B 5/14546; A61B 5/742; A61B 5/6831; A61B 5/6824; A61B 5/6833; A61B 2562/0271

(57) ABSTRACT

A device qualitatively and quantitatively detects blood analytes such as alcohol by passing sweat through a semi-permeable membrane to a reaction chamber. In the reaction chamber, the sweat can react with reagents that produce a color when reacted with the analyte. The presence of the color is used to determine that the analyte is present. The intensity of the color change can be measured to quantify the amount of analyte. The device can include tamper resistance elements to prevent the device from being removed.

### CROSS-REFERENCE TO RELATED APPLICATIONS

This application claims the benefit of U.S. Provisional Application No. 62/013,399, filed Jun. 17, 2014, which is hereby incorporated by reference in its entirety.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to devices for qualitative transdermal detection of analytes in tissue, particularly alcohol in human tissue.

2. Description of the Related Art

Underage alcohol consumption, as well as the over-consumption of alcohol, is a serious issue that leads to severe legal and health trouble, as well as a great deal of property damage every day. A device that can assess qualitatively and/or quantitatively how much alcohol an individual has consumed via sight or by cheap spectroscopy is needed.

Likewise a need exists for a device that detects certain date rape drugs could go further in ameliorating the problems that tend to occur in situations where alcohol is consumed. This would alert an individual or those around the individual that they have these drugs in their system, and lead them or others to seek help.

It has been shown that 2-8% percent of consumed alcohol is eliminated from the body via sweat, while the other 92-98% of alcohol is metabolized and excreted through other methods. It has also been shown that benzodiazepenes and other types of drugs can be detectable in sweat.

Devices exist for the evaluation of alcohol and other substances. However, these devices collect sweat. Then, the collected sweat must be sent to a lab for evaluation. By needing to be sent to a lab, the devices are not useful for making immediate, on-site decisions.

### BRIEF SUMMARY OF THE INVENTION

An object of the invention to provide a quick and inexpensive way to evaluate the blood alcohol content of an individual.

A further object of the invention is to provide a version of the device that cannot be detached from a wearer without adulterating the device. Other versions can be removable.

A further object of the invention is to provide a device that can quantify an amount (i.e. a concentration) of analyte in the wearer's blood. The analyte can be alcohol or a drug, for example, rohypnol.

The approximation of how much alcohol can pass through the skin can be estimated by Fick's law.

Flux=(Concentration₁−Concentration₂)(Area×Permeability Coefficient)/(Thickness)

In accordance with the objects of the invention, a device that is held in contact with the skin of a wearer is provided. Possible examples of the device include a bracelet and a bandage-like patch. The device measures blood alcohol levels transdermally. From the blood-alcohol levels, the consumption of alcohol can be correlated.

The device according to the invention will have a container with a semi-permeable membrane or a patch that will allow for the collection of alcohol, metabolites, and other substances from sweat. The semi-permeable membrane is located on the device so that is held against the skin of the wearer when the device is worn. The container will house either a chemical or an enzyme that will react with the analyte to produce a color change. The intensity of this color change will allow one either to assess visually how much alcohol a wearer has consumed or to assess spectrophotographically how much alcohol a wearer has consumed. An additional method for detection includes detecting an analyte electronically by measuring changes in conductance across the skin. An additional detection method includes detecting an analyte calorimetrically.

Additionally, the chemical version will allow for a temporary usage. The wearer can wear the chemical version for a certain pre-determined amount of time. The amount of alcohol consumed within a period can be determined visually by noting the degree of color change or by quantifying the amount of color with a spectrophotometer.

Other features of the invention are set forth in the appended claims.

Although the invention is illustrated and described herein as embodied in a device for detecting blood analytes transdermally, the invention is not limited to the details shown because various modifications and structural changes may be made without departing from the invention and the equivalents of the claims. However, the construction and method of operation of the invention together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a top side view of a strap embodiment according to the invention.

FIG. 2 is a bottom side view of the embodiment shown in FIG. 1.

FIG. 3 is a top side view of the embodiment shown in FIG. 1 while being worn by a user.

FIG. 4 is a front side view of the embodiment shown in FIG. 1.

FIG. 5 is a bottom side view of a bandage embodiment of the invention.

FIG. 6 is a top side view of the embodiment shown in FIG. 5.

FIG. 7 is a front side section view of the embodiment shown in FIG. 5.

FIG. 8 is a chemical formula depicting a reaction according to the invention.

### DETAILED DESCRIPTION OF THE INVENTION

Embodiments of the invention are described below and are shown in the figures of the drawing.

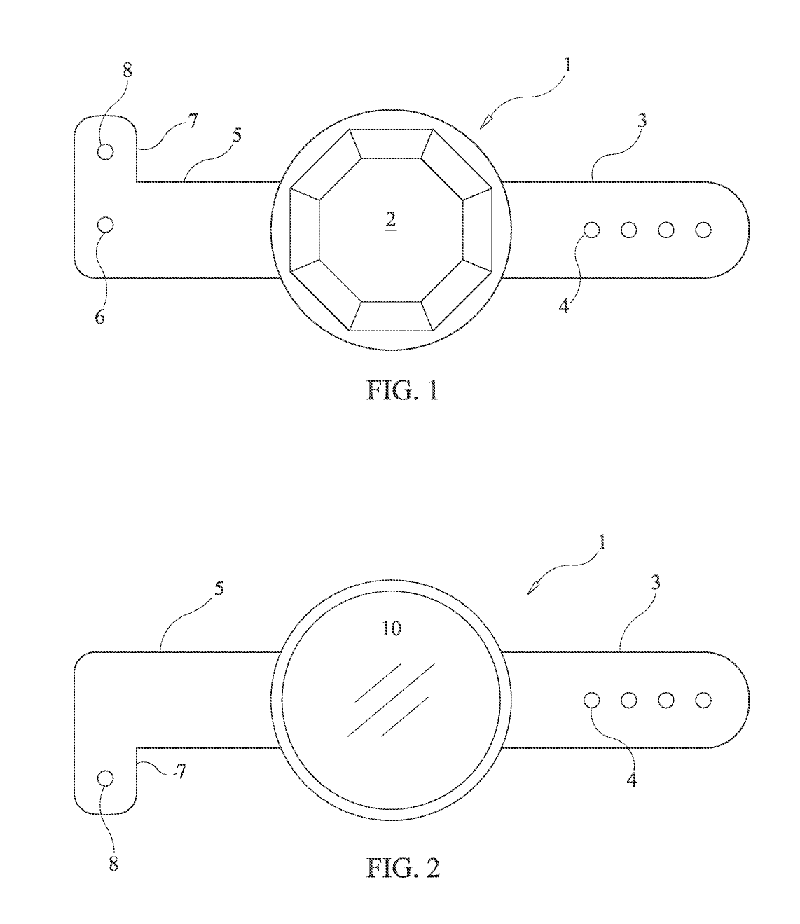

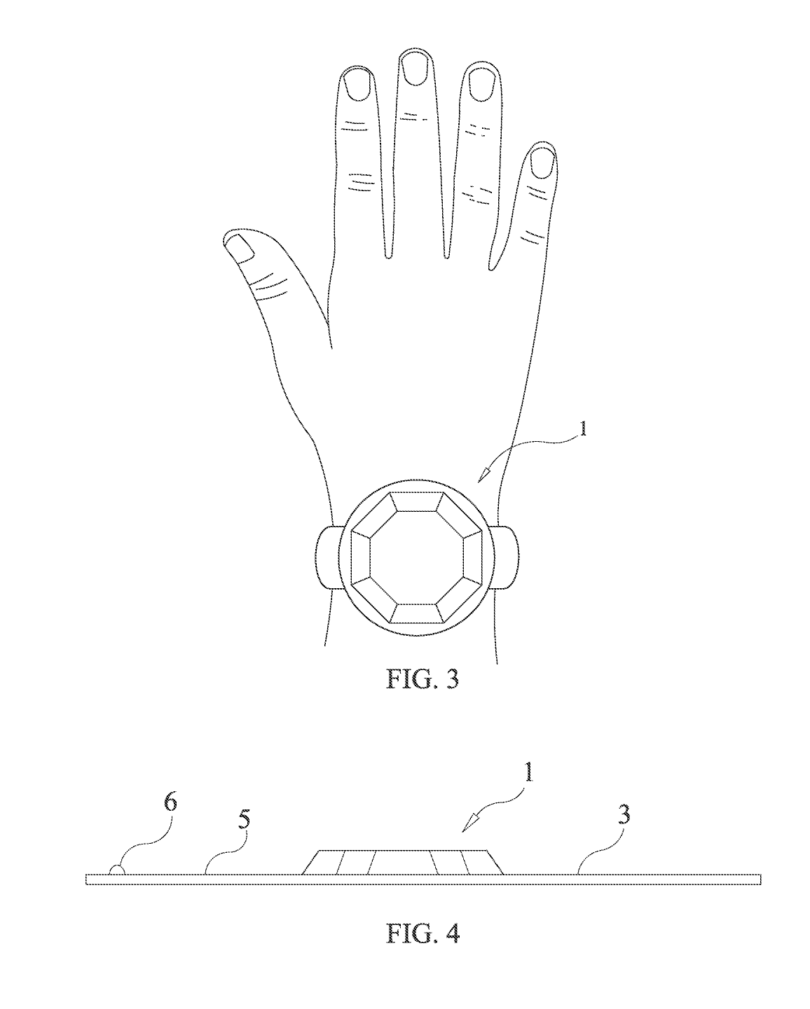

FIGS. 1-4 show a container embodiment of the invention. A container 1 holds a gel or liquid solution with either an enzyme that metabolizes alcohol, its metabolites, or other substances and causes a color change, or a chemical that will change colors after reacting with the above substances. The container 1 has a window 2. The window 2 allows the color of the contents of the container to be viewed. A first strap portion 3 has holes 4 formed therein. A second strap portion 5 has a peg 6 to be inserted in a hole 4. Tamperproof flap 7 has a hole 8 formed therein. After the peg 6 is inserted through the hole 4, the tamperproof flap 7 is folded over so that the hole 8 seats on the peg 6. Reopening the flap 7 destroys the flap and prevents re-closure.

In an embodiment that is not shown, the container 1 includes two subcontainers. In a first subcontainer reactants to detect a first analyte are included. In a second subcontainer, reactants to detect a second analyte are included.

A semipermeable membrane 10 forms a wall of the container 1. The semipermeable membrane 10 is located to rest against the skin of a wearer when the device is worn. The semipermeable membrane 10 is permeable to sweat and analytes. The semipermeable membrane is not permeable to the reactants held in the container 1. The semipermeable membrane 10 can have an adhesive on its outer surface to adhere the membrane to the skin of the wearer.

Potential chemicals/Enzymes for this product include but are not limited to the following: DMSO based reactions, Potassium dichromate, potassium permanganate, ADH enzyme, etc.

One potential construction of the device is enzymatic. The semi-permeable membrane on the bottom of the container allows alcohol and water to diffuse, but prevent enzyme and cofactors from leaving. The container can include pure alcohol dehydrogenase, diaphorase, zinc, and NAD+, and a tetrazolium salt. The NAD+ and zinc are necessary cofactors for alcohol dehydrogenase. In the presence of alcohol, alcohol dehydrogenase converts alcohol to aldehyde and NAD+ to NADH. In the presence of NADH, diaphorase converts tetrazolium salt to a colored formazan dye, and NADH into NAD+. The presence of color would then indicate the presence of alcohol in the container. The enzymes, cofactors, and associated chemicals can then be placed in capsules that can be broken immediately prior to use, when it is placed into solution inside the container.

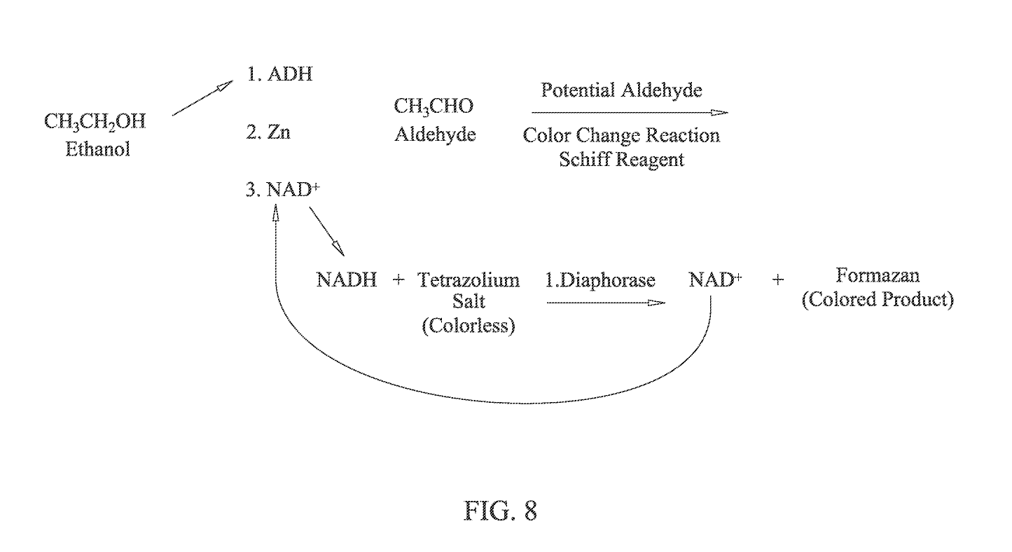

To improve sensitivity, an additional reagent could be added to cause a color change reaction with the aldehyde. FIG. 8 shows the general chemical equation of these reactions. Additionally, adding a way to convert the formazan back into tetrazolium salt could allow for a better real-time measurement of alcohol consumption.

A second potential construction would also be a container containing an oxidizing agent such as potassium chromate, ceric ammonium nitrate, or chromic acid in solution, gel matrix, or other medium. It would also have a membrane that would allow in water and alcohol, but prevent reagent from leaving. The alcohol would cause the reagents to undergo a color change that would report the presence of alcohol.

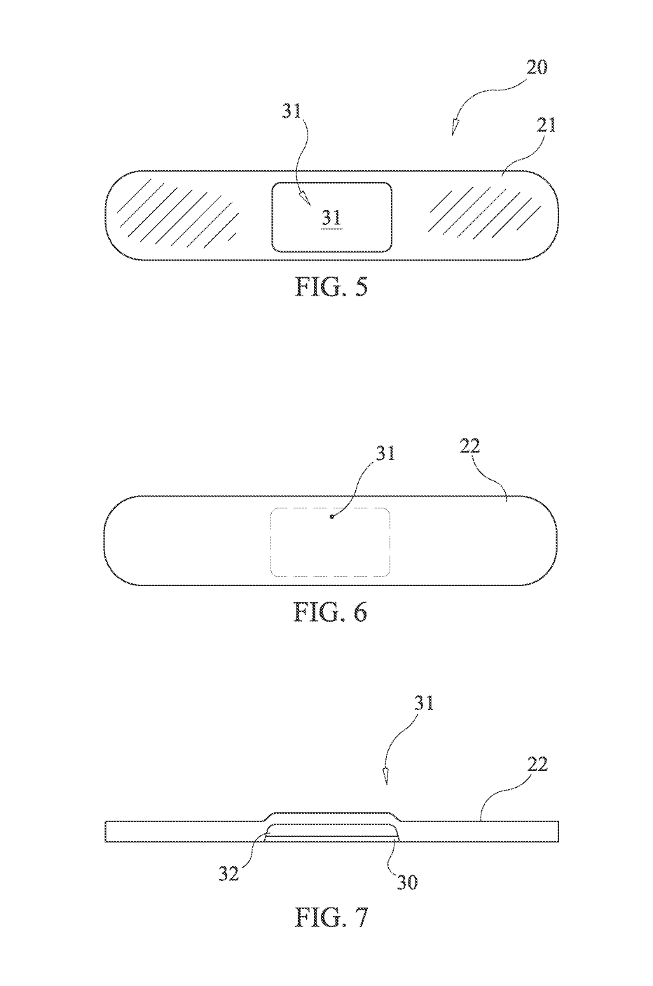

FIGS. 5-7 show a bandage embodiment. The bandage includes a plastic, non-permeable band 22. Adhesive 21 is disposed on the proximal surface of the band 22. A compartment 31 includes a semipermeable membrane 30 on its proximal surface. The compartment 31 includes a reaction chamber 32.

An embodiment which is not shown detects the presence of an analyte by indicating when electrical conductance across the skin reaches a threshold that is correlated to the presence of an analyte in the blood of the patient. When the threshold is reached, an LED light can be activated.

A unique identifier can be added to each device. Examples of identifiers include a barcode, ID number, or RFID tag. The unique identifier allows the device to be registered to a particular person to make the device more effective at tracking alcohol consumption and individuals.

What is claimed is:

1. A device for transdermally detecting an analyte in blood of a wearer, comprising:
a container having a hole formed therein;
a reactant being reactable with the analyte, said reactant being disposed in said container;
a semipermeable membrane covering said hole in said container, said semipermeable membrane being configured to contact skin of the wearer, said semipermeable membrane being permeable to the analyte and not permeable to said reactant, said semipermeable being configured to lay against skin of a wearer.

2. The device according to claim 1, wherein said reactant creates a photochemical change when reacted with the analyte.

3. The device according to claim 1, wherein said reactant is an enzyme.

4. The device according to claim 1, further comprising a conductance detector connected to said container, said conductance detector detecting a change in conductance created when said reactant reacts with the analyte.

5. The device according to claim 1, further comprising a calorimeter being connected to said container, said calorimeter detecting heat produced when said reactant reacts with the analyte.

6. The device according to claim 1, further comprising a tamperproof connector for connecting said container to the wearer, said tamperproof connector indicated when said container has been removed from the wearer.

7. The device according to claim 1, further comprising a strap connected to said container, said strap being configured to hold said semipermeable membrane on a wrist of the wearer.

8. The device according to claim 1, wherein the analyte is alcohol.

9. The device according to claim 8, wherein said reactant includes alcohol dehydrogenase, diaphorase, zinc, and NAD+, and a tetrazolium salt.

10. The device according to claim 1, further comprising an adhesive bandage, said adhesive bandage overlying said container and securing said semipermeable membrane against the skin of the wearer.
